(19) United States
(12) Patent Application Publication
(10) Pub. No.: US 2026/0306910 A1
(43) Pub. Date: Oct. 8, 2026

(54) GAMIFIED PHYSICAL THERAPY REHABILITATION SYSTEM

(71) Applicant: UNIVERSITY OF SOUTH FLORIDA, Tampa, FL (US)

(72) Inventors: Nathan D. Schilaty, Tampa, FL (US); John Templeton, Tampa, FL (US); Antonio Victor Gonzalez, Tampa, FL (US); Gabriel Mozombite Gallegos, Tampa, FL (US); Ashley Pham, Tampa, FL (US); Voon Hee Lee, Tampa, FL (US)

(21) Appl. No.: 19/639,114

(22) Filed: Apr. 3, 2026

Related U.S. Application Data
(60) Provisional application No. 63/782,893, filed on Apr. 3, 2025.

(51) Int. Cl. A63B 24/00; A61B 5/00; A61B 5/256; A61B 5/296; A61B 5/397; A63B 71/06; A63F 13/211; A63F 13/212; A63F 13/22; A63F 13/67

(52) U.S. Cl. CPC A63B 24/0075; A61B 5/256; A61B 5/296; A61B 5/397; A61B 5/4833; A61B 5/7465; A63B 71/0622; A63F 13/211; A63F 13/212; A63F 13/22; A63F 13/67; A63B 2024/0068; A63B 2024/0071; A63B 2024/0096

(57) ABSTRACT

Systems and methods are provided for improved patient/user compliance, adherence, and/or engagement with exercise regimens, including via interactive gamification and real-time sensing. For example, a gamified physical therapy system may include an EMG device with an EMG sensor to sense EMG signals representative of muscle engagement by a user. A wearable article may be dimensioned to be worn by the user at a first body position and to hold the EMG device at a position relative to a given target muscle. A memory may store user data including a user profile, exercise regimen parameters, and the EMG signals. Software may run to output an interactive digital game to a display, which may have an avatar and/or game elements corresponding to the exercise regimen parameters. The system may receive sensed EMG signals during the game, compute muscle engagement signals in real time, translate them into avatar control signals, and depict avatar movement relative to the game elements so as to convey performance feedback to the user in real time relative to the exercise regimen parameters. Session metadata, muscle engagement signals, and data indicative of performance are stored in a datastore which may be accessible by a provider.

### CROSS REFERENCE TO RELATED APPLICATIONS

This application claims priority to U.S. provisional patent application No. 63/782,893, filed on Apr. 3, 2025, the entire contents of which are incorporated herein by reference.

### STATEMENT OF GOVERNMENT SUPPORT

N/A

### BACKGROUND

Pediatric and adolescent physical rehabilitation for lower-limb conditions (including, for example, neuromuscular disorders such as muscular dystrophy, as well as post-injury and post-surgical recovery) involves asking the patients to engage in sustained, consistent, specific, and repetitive therapeutic exercises over extended periods. Traditional physical therapy approaches may be effective when performed consistently, but maintaining adherence and motivation, particularly in younger patients, presents a significant challenge. Younger patients may find repetitive exercise regimens monotonous and difficult to sustain without interactive or motivating feedback mechanisms.

Furthermore, traditional physical therapy (e.g., giving a patient a printout or video of exercises to perform) relies on patients to perform the exercises and accurately self-report their adherence. General training monitoring approaches, such as camera-based motion tracking or recording systems, have attempted to address guidance or tracking but do not capture granular, muscle-specific data and are often limited to only gross motion. As a result, such approaches may be limited in their ability to provide accurate, reliable, and precise information to a healthcare provider of muscle-specific engagement. Without this information, a trainer, physical therapist, or other provider would not be capable of data-informed personalization of therapy to each patient's specific neuromuscular profile and capability on any given day, to quantify the degree of muscle activation achieved during a therapeutic exercise, or to adapt therapeutic targets based on objective measures of individual patient performance.

Accordingly, there remains a need for rehabilitation systems and methods that overcome the disadvantages and obstacles currently facing many patients, such as lack of: (a) real-time, personalized monitoring of muscle activation during therapeutic exercise; (b) engaging, adaptive feedback mechanisms capable of motivating sustained and regimen-consistent participation by younger patients; and (c) provider-configurable parameters that enable a healthcare provider to individualize therapy targets, difficulty progression, and session structure based on each patient's clinical needs and capabilities.

### SUMMARY

The following presents a simplified summary of the disclosed technology herein in order to provide a basic understanding of some aspects of the present disclosure. This summary is, of course, not a comprehensive or extensive overview of the full content of the present disclosure nor of all perspectives or wording by which the novel, non-obvious, and useful subject matter hereof could be described or presented. It is not meant to specify any particular ‘key,’ ‘necessary,’ or ‘critical’ elements of the disclosed technology nor to delineate the scope of the present disclosure or any claims which might be based hereon. Rather, it is meant to present concise and ‘summary’ examples of how some concepts of the disclosed technology can be described.

In some aspects, the present disclosure can provide a method for providing guided therapeutic rehabilitation with improved patient interactivity and compliance. The method may comprise accessing stored patient exercise regimen data from a data storage, the patient exercise regimen data comprising at least a first gamification parameter, a first exercise motion, and at least a first EMG sensor position relative to muscles engaged by the first exercise motion. The method may further comprise confirming placement of a wearable EMG device on a patient's limb at the first EMG sensor position, and receiving a real-time readout of measurement data from the EMG device. A digital game environment may be communicated to a display screen for viewing by the patient, including at least one instruction, the digital game environment having exercise gamification elements configured to influence the patient to perform exercises according to the patient exercise regimen data. The method may further comprise computing change features in the readout of measurement data and transforming the change features into game control signals corresponding to a patient movement characteristic, changing at least one game element in the digital game environment according to the game control signal, and displaying to the patient in real time, using the exercise gamification elements, feedback information comparing performance of the exercises relative to the patient exercise regimen data.

In other respects, the present disclosure provides a gamified physical therapy system comprising an EMG device having at least one EMG sensor configured to sense EMG signals representative of muscle engagement by a user and a data link to a processor to communicate the EMG signals to the processor in real time. The system may further comprise a wearable article dimensioned to be worn by the user at a first body position and having a compartment for the EMG device so as to position the EMG device relative to a given target muscle of interest when the wearable article is worn at the first body position, and a memory comprising a datastore configured to store user data including a user profile, an exercise regimen, and the EMG signals. The memory may further comprise software instructions which, when executed by the processor, cause the system to output an interactive digital game to a user interface including a display, the game having an avatar and game elements defined according to parameters of the exercise regimen, receive the sensed EMG signals during the game and compute muscle engagement signals from the sensed EMG signals in real time, translate the muscle engagement signals into avatar control signals, and depict movement of the avatar in the game on the display in real time according to the avatar control signals, wherein depiction of movement of the avatar is relative to the game elements so as to communicate performance feedback to the user in real time relative to the exercise regimen. The software instructions may further cause the system to store in the datastore metadata concerning use of the game, the muscle engagement signals, and data indicative of performance.

### BRIEF DESCRIPTION OF THE DRAWINGS

The foregoing features of embodiments will be more readily understood by reference to the following detailed description, taken with reference to the accompanying drawings, in which:

FIG. 1 is a diagram illustrating an example system architecture for a gamified physical therapy rehabilitation system according to some embodiments.

FIG. 2 is a diagram illustrating an example wearable muscle-activity sensing apparatus according to some embodiments.

FIG. 3 is a flow diagram illustrating an example electromyography signal processing pipeline according to some embodiments.

FIG. 4A is an interface diagram illustrating an example calibration menu of a gamified therapeutic application according to some embodiments.

FIG. 4B is an interface diagram illustrating example calibration instruction screens of a gamified therapeutic application according to some embodiments.

FIG. 4C is an interface diagram illustrating example gameplay and level completion screens of a gamified therapeutic application according to some embodiments.

FIG. 5 is a flowchart illustrating aspects of a gamified exercise system according to some embodiments.

FIG. 6 is a diagram illustrating an example wearable article with ports to guide sensor placement according to some embodiments.

### DETAILED DESCRIPTION

The following description will provide a disclosure of various features, approaches, and aspects of example systems and methods that can overcome the limitations described above, and allow for more effective, personalized, and engaging rehabilitation through real-time muscle-activity monitoring, adaptive gamified biofeedback, and provider-configurable therapy parameters. First, a general description will be provided of aspects of technologies that may be utilized in systems and methods of the present disclosure. Second, an overview of illustrative system/hardware architectures will be provided along with an overview of a framework for deploying certain processes and algorithms of the present disclosure. Third, a description of the inventors' experiments and validation studies will be provided.

Described here are systems and methods directed to gamified physical therapy rehabilitation, and more particularly to wearable electromyography-based sensing systems integrated with adaptive, interactive game interfaces for promoting sustained patient engagement in therapeutic exercises and enabling provider-configurable, personalized rehabilitation protocols.

The present disclosure will now provide overview descriptions of various approaches to deploying embodiments contemplated herein. It should be understood that the processes and algorithms described below are not limiting of the scope of this disclosure, can be combined in various configurations (in whole or in part), and may be adapted to replace, complement, and/or fit with attributes and needs of different training or therapeutic contexts and deployment environments, including but not limited to pediatric and adolescent lower-limb rehabilitation, post-surgical musculoskeletal recovery, neuromuscular disorder management, upper-extremity rehabilitation, athletic training and return-to-sport assessment, at-home patient-directed therapy, clinical in-office therapy sessions, school-based adaptive physical education programs, and remote provider-supervised telerehabilitation.

### Example Hardware Configurations

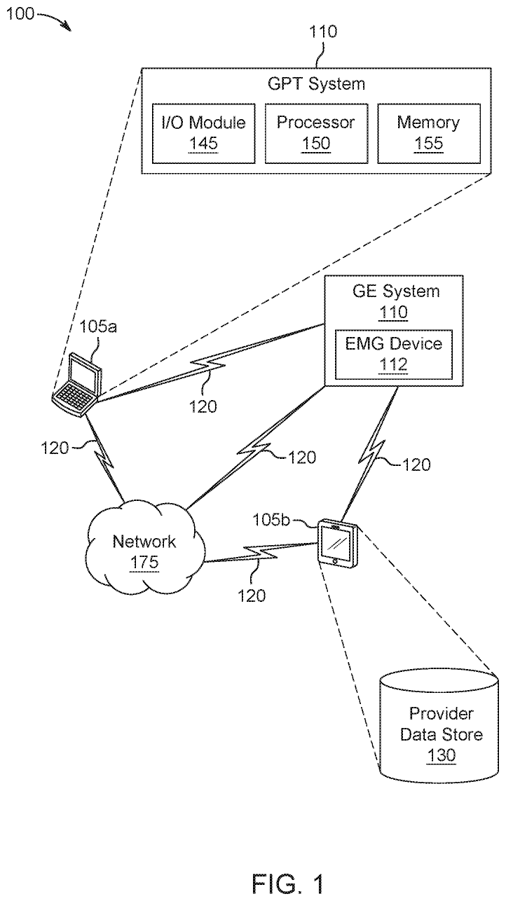

FIG. 1 is a schematic diagram of an example computing environment that supports a gamified exercise or physical therapy system in accordance with one or more aspects of the present disclosure. The network environment 100 may include one or more computing devices 105-a through 105-b (collectively referred to as computing devices 105), at least one gamified exercise system 110 having an electromyography (EMG) device 112 connected to a network, and at least one provider data store database 130. The Gamified exercise system 110 connected to a network may be in communication with the one or more computing devices 105 via one or more communication links 120, which may also include other communication networks, such as classical networks and wired networks. The communication links 120 may be established using any suitable hardware, firmware, and/or software for communicating information over one or more communication networks, including local, wide-area, private, public, or hybrid networks. For example, the communication links 120 may utilize hardware, firmware, and/or software that can be used to establish a Wi-Fi connection (including IEEE 802.11ax/Wi-Fi 6 or Wi-Fi 6E), a Bluetooth or Bluetooth Low Energy (BLE) connection, a cellular connection (e.g., a 4G LTE, 5G NR, or future-generation network complying with any suitable standard, such as CDMA, GSM, LTE Advanced, NR, or mmWave 5G), a Zigbee, Z-Wave, Thread, or other low-power mesh network connection, a LoRa or LoRaWAN connection for long-range low-power applications, a wired serial connection (e.g., USB or UART), a wired Ethernet connection, or any combination thereof. In some embodiments, the network environment 100 may participate in a multi-tier architecture in which data is communicated among on-device edge processing, local fog or gateway nodes, and remote cloud servers or data centers, with different processing tasks allocated across tiers based on latency, bandwidth, privacy, and computational resource constraints. In some embodiments, one or more components of the gamified exercise system 110 may communicate via a wired serial link-for example, a wireless shield or wireless transceiver module associated with the EMG device 112 may transmit EMG data wirelessly to a microcontroller (e.g., an ESP32-WROOM or similar Wi-Fi-and Bluetooth-capable microcontroller), which in turn communicates with a computing device 105 via a serial connection. The one or more computing devices 105 may be in communication with each other via the at least one link or they may directly communicate via peer-to-peer or sidelink communications, for example.

The one or more computing devices 105 may be any type of computing device, such as classical computing devices, such as a desktop computer, laptop, mobile phone, smartphone, tablet, database, personal digital assistant (PDA), tablet, personal computer, a workstation, a mainframe computer, a server, or other electronic device. Depending on the deployment context, the one or more computing devices 105 may also take the form of a cloud-based server or cluster, an on-premise workstation or server, an edge computing node, a wearable device, an embedded system, or a distributed network of any combination of the foregoing. In some embodiments, a computing device 105 may operate as a standalone unit; in other embodiments, it may function as one node within a larger system architecture comprising client devices, edge nodes, fog computing layers, cloud resources, and/or remote servers communicating over one or more networks. The one or more computing devices 105 may be enabled with Internet or other network access and may be locatable by the at least one network, such as the networks described herein. In one aspect, the one or more computing devices 105 may be configured to transmit instructions to control various devices, such as to receive data or other signals from external devices, and transmit the received data or other data to the gamified exercise system 110, the at least one provider data store database 130, a server, or another remote computing device 105 (e.g., another database) of the network environment 100.

In the example of FIG. 1, the computing devices 105-a may include an Input/Output (I/O) module 145, a processor 150, and a memory 155. Here, the I/O module 145 may receive input and output data from the gamified exercise system 110. The processor 150 may be any suitable hardware processor or combination of processors, such as a central processing unit (CPU), a graphics processing unit (GPU), a neural processing unit (NPU), an application-specific integrated circuit (ASIC), a field-programmable gate array (FPGA), a digital signal processor (DSP), a microcontroller (MCU), a system-on-chip (SoC), or any other suitable processing unit or combination thereof. In embodiments involving edge deployment, low-power inference, or miniaturized form factors (e.g., wearable devices or embedded sensors), the processor 150 may be a resource-constrained microcontroller or a dedicated low-power accelerator optimized for on-device processing with minimal energy consumption. In some embodiments, the processor 150 may comprise a microcontroller such as an ESP32-WROOM or similar Wi-Fi-and Bluetooth-capable microcontroller suitable for receiving and relaying EMG data from the EMG device 112 to a computing device executing the gamified application. The processor 150 may be capable of executing instructions stored in memory 155 to perform signal processing operations, including root mean square (RMS) filtering of raw EMG data from the EMG device 112, moving average smoothing, proportional-integral-derivative (PID) control calculations, calibration protocol execution, game logic processing, real-time rendering of visual feedback elements, threshold comparisons, data logging operations, and coordination of communication between the I/O module 145 and memory 155. In some embodiments, the gamified application may be implemented in a programming language such as Python and may utilize a game development library such as PyGame for rendering visual feedback and managing game state in real time. The memory 155 may include any suitable volatile memory, non-volatile memory, storage, or any suitable combination thereof, including random access memory (RAM), read-only memory (ROM), electronically-erasable programmable read-only memory (EEPROM), one or more flash drives, one or more solid-state drives (SSDs), embedded flash memory, or on-chip memory integrated with the processor 150. The memory 155 may store computer-readable, computer-executable, or processor-executable code that, when executed by the processor 150, causes the computing device 105 to perform one or more functions described herein. The code may be stored in a non-transitory computer-readable medium such as the memory 155 or another type of memory. In some cases, the memory 155 may include, among other things, a basic input/output system (BIOS) which may control basic hardware or software operation such as the interaction with peripheral components or devices. The code stored in the memory 155 may include software for at least one gamification module, at least one clinic configuration module, and other components described herein. It should be understood that code for performing the functions described herein may reside on one or more of the computing devices 105 within the network environment 100. For example, code for signal acquisition and wireless transmission may reside on a processor or microcontroller associated with the EMG device 112, code for the gamified application and signal processing pipeline may reside on a computing device 105-a serving as the patient-facing game device, and code for provider configuration and data analytics may reside on a computing device 105-b associated with the provider data store database 130, or any combination thereof. The memory 155 may be capable of storing raw and processed EMG signal data, user calibration profiles, including maximum activation values and smoothness metrics, personalized threshold parameters, game state information, including current level, score, and lives remaining, historical performance data across multiple sessions, executable code for the signal processing module, control translation module, personalized behavior adaptation module, gamification engine module, clinic configuration module, and data logging module, configuration parameters set by providers, including target activation levels and repetition goals, and temporary buffers for real-time signal processing operations. The computing devices 105-a may further include a user interface, which may include any suitable combination of input and output components for interaction between the system and a user, operator, provider, or other human, such as a touchscreen, a liquid crystal display (LCD), an organic light-emitting diode (OLED) display, an augmented reality (AR) or mixed reality (MR) overlay, speakers, haptic actuators, microphones, or motion sensors. In embodiments directed to wearable devices or other miniaturized form factors, the user interface may be minimal or absent entirely, with outputs communicated instead to a companion mobile application, a clinical monitoring platform, or a remote dashboard accessible via a network connection.

The gamification module stored in memory 155 may manage interactive gameplay elements, progression structures, and feedback mechanisms designed to sustain user engagement throughout therapeutic exercises. The computing device 105-a executing the gamified application may be any suitable user-facing device, including a laptop computer, desktop computer, mobile phone, smartphone, tablet, or other portable computing device. In some embodiments, the gamified application may be loaded into a television-based application environment (such as a Roku platform, Amazon Fire TV, Apple TV, a native smart television application framework, or other streaming or application-capable television device) and the EMG device 112 may communicate with the television directly or via a wireless dongle or bridge device connected to the television. In further embodiments, the EMG device 112 may connect via Bluetooth or other wireless protocol to a dedicated gaming console, such as a Nintendo Switch or similar gaming environment, provided that a compatible game application implementing the signal processing, calibration, and gamified biofeedback functionality described in this specification is loaded onto the gaming console.

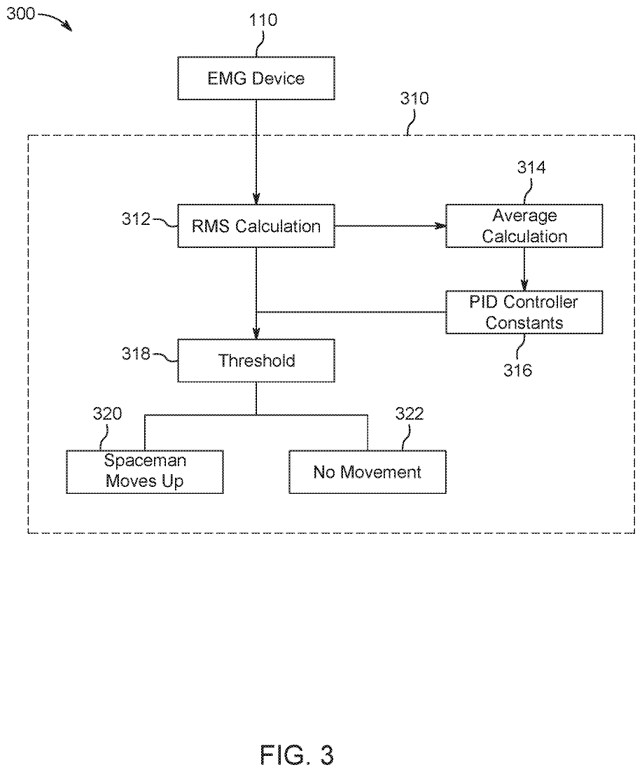

The gamification module may control or affect the presentation of visual stimuli that causes the patient user to move or tense their muscles in ways that meet the prescribed PT goals (e.g., duration, intensity, range of motion, fatigue, eccentric, etc.). For example, the patient may understand from the gaming environment that their muscle movements control their character (astronauts, vehicles, animals, people, or other themed avatars) as it interacts with or moves through environmental graphics that contain obstacles, targets, mazes, adversaries, etc. The gamification module may interface with the signal processing pipeline 310 described with respect to FIG. 3 to receive control signals derived from EMG data and translate these signals into character movements, velocity changes, or other responsive behaviors that provide intuitive biofeedback to users during exercise performance. In other embodiments, IMU and/or optical/depth sensors may be utilized in conjunction with the EMG data to encourage even more particularized dynamic muscle engagements, in which both strain and motion are performed and measured.

The particularized presentation of the environmental elements can, with the knowledge that the user will want to advance through the game due to the gamification design, encourage or require the patient to perform very precise movements in consistent and accurate ways according to their PT regimen. For example, a given game operated via the gamification module may entail space-themed backgrounds with planets, stars, and celestial objects, obstacle elements such as floating islands or asteroids that users must navigate, and animated effects responsive to user actions. To further encourage user behavior within specified parameters, the gamification module may implement game logic including collision detection algorithms that determine when the character contacts obstacles or successfully passes through target zones, scoring systems that award points based on successful navigation or sustained activation duration, level progression mechanics that unlock subsequent stages upon completion of prerequisite levels, and multi-life systems that allow users a predetermined number of attempts before requiring level restart. Additionally, the gamification module may coordinate audio feedback, including background music, sound effects triggered by threshold crossings or collisions, and auditory cues indicating level completion or failure.

To determine how to utilize game elements to achieve PT goals, the gamification module may further access or manage configurable gameplay parameters that correspond to therapeutic variables. For example, the number of obstacle elements or logic gates presented within a given level may correspond to a prescribed number of muscle activation repetitions, such that a provider-specified repetition count is reflected in the quantity of obstacles the user must navigate. The horizontal length of obstacle elements along the direction of travel may define the duration for which the user must sustain muscle activation above a threshold in order to successfully pass through or alongside the obstacle, thereby mapping obstacle length to sustained contraction duration as a therapeutic parameter. The vertical proximity between adjacent obstacles or passage zones may define the precision of muscle activation control required, with closer spacing demanding finer modulation of contraction intensity. Thus, the gamification module may further promote consistency in muscle activation quality, rather than mere detection of gross motion, by providing continuous proportional feedback that rewards sustained, controlled contractions at prescribed intensity levels.

The gamification module may further implement configurable temporal spacing between successive obstacle elements or activation-requiring events within a given level, defining rest intervals during which the user is not required to maintain muscle activation above the threshold. The duration of such rest intervals may be specified by the provider via the clinic configuration module or may be set to a default value (e.g., approximately ten seconds between successive obstacles). Configurable rest intervals are of particular relevance in therapeutic contexts involving patients with conditions characterized by accelerated muscle fatigue, such as Duchenne muscular dystrophy, where insufficient recovery time between successive contractions may result in premature exhaustion, diminished performance, and reduced therapeutic benefit. By adjusting the temporal spacing between obstacles, the provider may control the duty cycle of muscle activation within a session, balancing the therapeutic goal of repeated activation against the patient's fatigue tolerance.

The gamification module may also apply a handicap coefficient or calibration scaling factor derived from the user's calibration profile, including maximum activation values and smoothness metrics, to scale gameplay difficulty relative to the individual user's capability, ensuring that patients with differing baseline abilities receive appropriately challenging gameplay without manual difficulty adjustment.

In some embodiments, the gamification module may include a cognitive training mode in which reaction time, decision-making speed, or dual-task performance requirements are layered onto the EMG-driven gameplay, targeting cognitive readiness in addition to muscular rehabilitation. Such cognitive training modes may be of particular relevance in sport-specific return-to-play assessment contexts, where cognitive and motor demands are simultaneously evaluated. In some cases, the gamification module may incorporate randomization or procedural generation elements to provide variety across repeated sessions while maintaining consistent therapeutic requirements, such as varying the vertical spacing between obstacles within defined ranges or altering the visual appearance of environmental elements while preserving their functional properties.

The gamification module may support multiple game types or themes selectable by the user, each presenting a distinct visual environment and control mapping while drawing from the same underlying EMG signal processing and therapeutic parameter framework.

The clinic configuration module may comprise a provider-side configuration interface and a patient-side adaptation module. The provider-side configuration interface may be accessible to authorized healthcare providers, such as physical therapists or providers, via a computing device 105-b or other authorized device, and may present user interfaces through which providers can specify therapeutic parameters for individual patients. In some embodiments, the provider-side configuration interface may accept parameter inputs at two levels of abstraction: first, the provider may directly specify gameplay parameters, such as target muscle activation thresholds expressed as percentages of the patient's maximum voluntary contraction determined during calibration or as absolute EMG amplitude values in microvolts, repetition counts indicating the number of successful threshold crossings or obstacle navigations required per session, spatial parameters of gameplay elements such as the vertical proximity between obstacles to modify fine motor control requirements or the horizontal width of passage zones to adjust difficulty, session duration targets or time limits for individual levels or complete therapeutic protocols, and selection of therapeutic modes such as strength training with progressively increasing thresholds, endurance training with sustained activation requirements, or maintenance protocols with stable difficulty levels; and second, the provider may specify clinical rehabilitation parameters using the clinical parlance with which the provider is already familiar, including target muscle groups, desired range of motion, contraction type (e.g., concentric, eccentric, or isometric), target activation intensity relative to a clinical baseline, prescribed exercise movements, repetition and set goals, rest intervals, and session frequency. In some embodiments, the translation of clinical rehabilitation parameters into corresponding gameplay parameters may be performed dynamically by the patient-side adaptation module or the gamification module on the computing device 105-a. Because the gamified application may support multiple game types or themes selectable by the patient, and because the manner in which a clinical concept such as “eccentric contraction” maps to gameplay mechanics may differ across game types, the computing device 105-a may dynamically determine the appropriate gameplay parameter mapping based on the game type currently selected by the patient. For example, a provider prescription specifying eccentric contractions may be translated into a first set of obstacle configurations and control mappings in a first game type and into a different set of obstacle configurations and control mappings in a second game type, with the translation performed at runtime by the computing device 105-a based on a mapping engine or rules framework associated with each game type. In other embodiments, the mapping between clinical rehabilitation parameters and gameplay parameters may be pre-programmed within the clinic configuration module, such that the provider-side configuration interface stores a predetermined association between each clinical term or parameter and a specific instruction or parameter value for each game type known to the system. In such embodiments, when a provider specifies a clinical parameter such as “eccentric,” the clinic configuration module may resolve the corresponding gameplay parameter for the patient's selected or assigned game type and transmit the resolved gameplay parameter to the computing device 105-a without requiring the patient device to perform the translation. In some cases, the provider-side configuration interface may include preset protocol templates for common conditions, such as post-ACL reconstruction rehabilitation, cerebral palsy management, or muscular dystrophy maintenance, that providers can select and further customize based on individual patient needs and capabilities. The provider-side configuration interface may implement secure authentication mechanisms requiring provider credentials, such as usernames and passwords, biometric verification, or multi-factor authentication, to ensure that only authorized healthcare providers can modify therapeutic parameters or access patient data. Additionally, the provider-side configuration interface may provide data visualization tools, including graphs displaying EMG activation trends over multiple sessions, adherence dashboards showing completion rates relative to prescribed frequency, comparative analytics presenting current performance against baseline measurements or population norms, and alert configuration interfaces allowing providers to define thresholds that trigger automated notifications when patient adherence falls below acceptable levels or performance metrics indicate plateau or regression requiring intervention. The patient-side adaptation module may reside on the computing device 105-a executing the gamified application and may be configured to access current and updated provider-prescribed instructions, whether stored locally or retrieved from the provider data store database 130 via a network connection, and to adapt and modify gameplay parameters accordingly. For example, when a provider updates a patient's prescribed activation threshold, repetition target, or therapeutic mode via the provider-side configuration interface, the patient-side adaptation module may retrieve the updated parameters at the start of the next session or, in some embodiments, during an active session, and may automatically adjust the gamification module's difficulty settings, obstacle configurations, level progression requirements, and scoring criteria to conform to the updated therapeutic regimen without requiring manual reconfiguration by the patient or caregiver.

In some embodiments, the provider-side configuration interface may support real-time modification of therapeutic parameters during an active patient session. For example, a provider observing a patient's session, whether in person or via a remote monitoring interface, may adjust activation thresholds, obstacle configurations, difficulty levels, or other gameplay parameters while the patient is actively engaged in gameplay, with the modified parameters taking effect immediately or at the next level transition without requiring the patient to terminate and restart the session. Real-time parameter modification may enable the provider to respond dynamically to observed patient performance, such as reducing difficulty if the patient exhibits signs of excessive fatigue or frustration, or increasing difficulty if the patient is completing levels with minimal effort.

In some embodiments, the system may be integrated with telehealth or tele-training platforms to enable remote provider observation and supervision of patient sessions. For example, the computing device 105-a may transmit real-time or near-real-time session data, including live EMG activation levels, gameplay state, and performance metrics, to the computing device 105-b via the network 175, enabling a provider to observe the patient's session remotely and provide guidance, encouragement, or parameter adjustments without requiring the provider and patient to be co-located. The transmitted session data may be incorporated into a telehealth visit interface, allowing the provider to review objective EMG-based performance metrics alongside the patient's subjective reports during a virtual consultation. Such integration may be of particular value in at-home deployment contexts where patients perform therapy sessions independently between periodic telehealth check-ins with their provider.

The computing devices 105-b may include access to at least one provider data store database 130. The provider data store database 130 may be configured to store patient records, including demographic information, diagnosis codes, and assigned therapeutic protocols, aggregated performance data from multiple sessions, including raw and processed EMG signal values, activation amplitude trends, muscle fatigue indicators, and session-to-session progression metrics, provider-configured therapeutic parameters for individual patients, including target muscle activation thresholds expressed as percentages of maximum voluntary contraction or absolute amplitude values. Additionally, in some examples, the at least one provider data store database 130 may be capable of prescribing various uses for the gamified exercise system 110, such as repetition counts or sustained contraction durations, spatial configuration parameters defining obstacle proximity and size within gameplay, session duration targets, and selected therapeutic modes, such as strength training.

Various uses of the provider datastore are also contemplated. The datastore may maintain data involving endurance training or maintenance protocols, as well as historical calibration data tracking changes in patient maximum activation capability, control smoothness metrics, and handicap coefficients over the course of treatment. Timestamped session logs may indicate when patients initiated and completed exercise sessions, total engagement duration, level completion status, and adherence rates relative to prescribed frequency. Comparative analytics may enable visualization of patient progress relative to baseline measurements, previous time periods, or anonymized population norms for similar conditions and demographics. Access control records may define which authorized providers or healthcare providers may view, modify, or export data for specific patients in compliance with privacy regulations. Alert configuration settings may trigger automated notifications to providers when patient adherence falls below specified thresholds, performance metrics indicate plateau or regression, or anomalous EMG patterns suggest equipment malfunction or improper electrode placement. Audit logs may record all data access, modification, and export activities for security and compliance purposes. The EMG device 112 may comprise a surface EMG sensor module, such as a MyoWare 2.0 Muscle Sensor or similar biosensor, configured to detect superficial EMG activity via disposable gel electrodes. In some embodiments, the EMG device 112 may comprise two signal detection electrodes and one reference electrode arranged in a triangular or bipolar electrode configuration. The EMG device 112 may further comprise a wireless transmission module, such as a MyoWare 2.0 Wireless Shield or similar wireless shield, configured to transmit acquired EMG data wirelessly to a microcontroller or computing device. The EMG device 112 may be housed within a wearable sleeve or garment, such as a double-layered neoprene sleeve with an inner layer contacting the skin and an outer layer providing structural support, configured to securely hold the EMG sensing components in proper alignment with the target muscle group during therapeutic exercises. Thus, an arrangement or relative configuration of sensors of the EMG sensor in the EMG device 112 may match an arrangement or relative configuration of ports, holes, grommets, or other skin access points in the wearable article that holds the EMG device. In further embodiments, the electrode access points and/or the compartment of the wearable article may be positioned relative to the intended wearing orientation of the article, such that some or all (e.g., two) electrodes align along a contraction axis or muscle fiber direction of a target muscle of interest.

Hook-and-loop fasteners, elastic bands, or other adjustable closure mechanisms may be integrated to accommodate different limb circumferences and ensure a snug, secure fit that minimizes motion artifact during dynamic movements. In addition to the EMG device 112, the gamified exercise system 110 may incorporate one or more supplemental sensors, which may include inertial measurement unit (IMU) sensors such as accelerometers, gyroscopes, and magnetometers for capturing limb orientation and movement dynamics, force or pressure sensors for detecting contact or weight-bearing status, optical or infrared sensors for proximity detection, or GPS modules for location-based session tracking in remote or at-home deployment contexts.

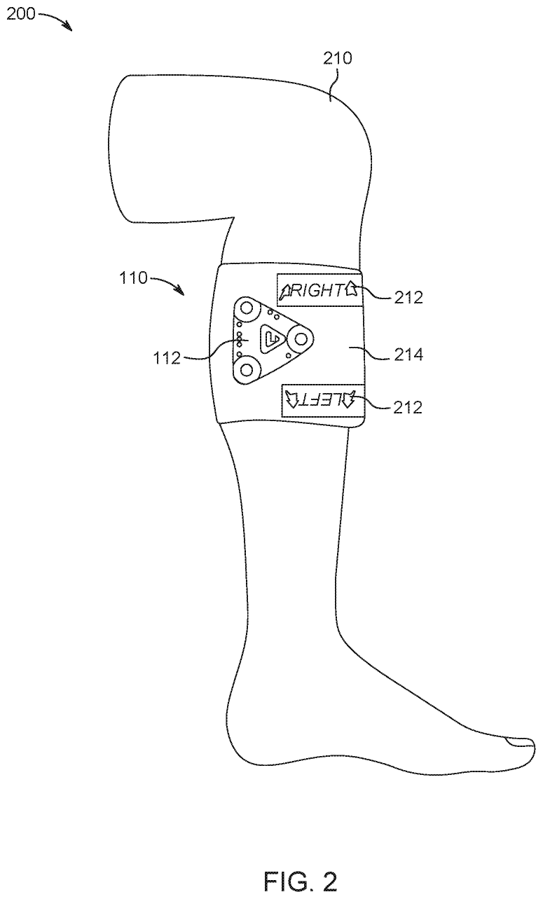

FIG. 2 illustrates an example limb system 200 using a limb-adapted gamified exercise system 110. The system includes a wearable article 214 dimensioned to fit about a body location (in the example shown, the body location is a lower leg 210 of a user, with a target muscle that is a calf muscle as depicted). The wearable article 214 may be formed of an athletic or fitness type fabric, such as a dry fit or evaporative synthetic fabric, and may be an elastic and stretchable fabric (such as neoprene, spandex, etc.) so as to allow for compression of an EMG sensor against the user's body location. The article 214 also includes a compartment (such as a holster, pocket, latch, snap, opening, strap, or other attachment or affixation mechanism) to hold an EMG device 112 at a given muscle location when the wearable article 214 is affixed about the user's leg/calf (as one example body location, according to aspects of the present disclosure. The gamified exercise system 110 may include one or more labels 212 for installation assistance, such as being printed visual onto the wearable article. In other embodiments, the labels 212 may be physical objects having curvature, nubs, openings, etc. that guide a user to wear or affix the wearable article in a given location at a given orientation, such as about a kneecap or patella, as an example.

As shown in FIG. 2, the EMG device 112 is positioned on the lower leg of patient 210 around the calf region. In the example of FIG. 2, the gamified exercise system 110 is positioned where the gastrocnemius muscle is located to collect data using the device 112. Labels 212 may provide visual indicators to assist users in proper device orientation and placement on the limb. In various examples, the labels 212 may include directional arrows indicating “RIGHT” and “LEFT” to denote which limb the device should be worn on, or alternatively, upward-facing arrows that allow the same device to be reversibly configured for use on either the right or left limb by rotating the device 180 degrees.

The device 112 comprises a wearable sleeve or garment configured to securely hold EMG sensing components in proper alignment with the target muscle group during therapeutic exercises. The gamified exercise system 110 may be fabricated from elastic or form-fitting materials such as neoprene that conform to the contours of the patient's limb while providing sufficient compression to maintain stable electrode contact with the skin. Within some examples, adhesive tabs may be used in combination with the device. In the example shown, device 112 is worn around the calf region below the knee and above the ankle, positioning the EMG sensors over the gastrocnemius muscle. The gamified exercise system 110 may incorporate a double-layered construction with an inner layer contacting the skin and an outer layer providing structural support and housing for electronic components. Hook-and-loop fasteners, elastic bands, or other adjustable closure mechanisms may be integrated into the gamified exercise system 110 and its hardware to accommodate different limb circumferences and ensure a snug, secure fit that minimizes motion artifact during dynamic movements.

Additionally, labels 212 serve as visual guides to facilitate correct device placement and orientation by patients or caregivers. Proper electrode placement aids in obtaining clean EMG signals, as electrodes must be positioned along the muscle fiber direction and at appropriate distances from motor points and tendon insertions. The labels 212 may include text, symbols, arrows, or color-coded markings that indicate the correct rotational orientation of the device relative to anatomical landmarks. In certain examples, labels 212 may be positioned on the exterior surface of the device 112 such that when the device is worn correctly, the labels are visible to the user or an assistant helping with device application.

While FIG. 2 depicts the device 112 positioned on the lower leg, it should be understood that the form factor and labeling approach illustrated may be adapted for placement on other body regions including the upper leg (quadriceps or hamstring muscles), upper arm (biceps or triceps muscles), lower arm (forearm flexors or extensors), or other locations where surface EMG signals can be reliably acquired from target muscle groups. The modular nature of the device design allows the same core sensing and transmission components to be integrated into different garment configurations, such as sleeves or straps sized for different anatomical regions, for specific anatomical sites while maintaining the installation assistance features provided by labels 212. In some embodiments, the system may automatically detect or allow user selection of which muscle group is being targeted based on the form factor of the sleeve or garment in use, a device identifier associated with the sleeve, or user input at the start of a session, and may load appropriate calibration profiles, threshold parameters, and game configurations accordingly, so that a patient transitioning between different anatomical targets need not manually reconfigure the system's signal processing or gameplay parameters.

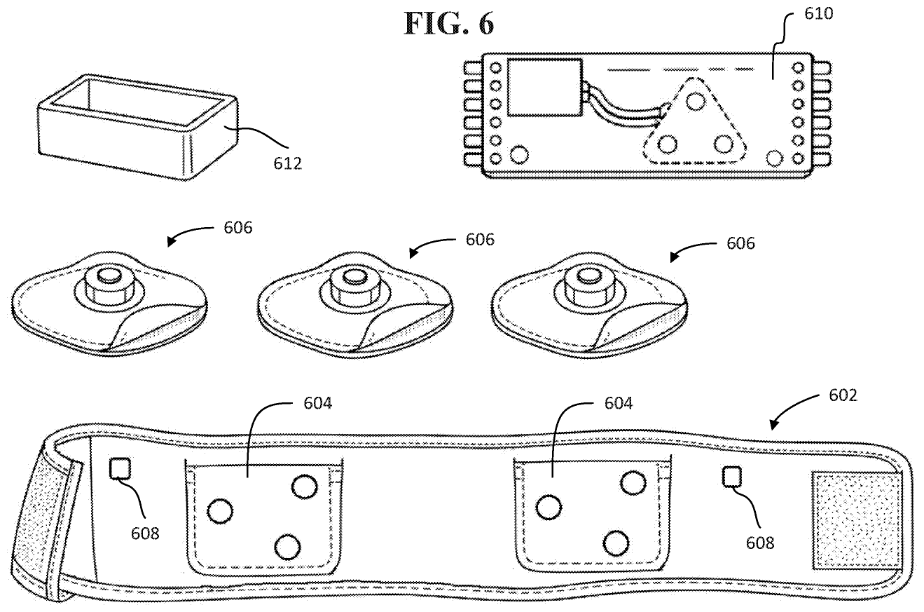

FIG. 6 shows alternative design aspects of an EMG device 112. For example, a wearable article 602 may be dimensioned as a wrap, to be affixed about a body location including an arm, leg, or torso. As shown, the article 602 has two compartments 604 (e.g., pockets or other holding elements as described above) which contain ports in specific configurations such that, when the wrap is worn, the relative locations of the ports will cause electrodes (such as 606) to align with muscle fibers along an axis of interest of a target muscle. As shown, the two compartments 604 would allow for EMG measurement of two muscles, such as around a joint or along a spine. The article also contains integrated IMU devices 608 (such as gyroscopes, accelerometers, or similar devices) to provide further information about how the wrap is being worn. Further, the wrap can also be configured to allow for customized placement of electrodes, such as when the compartments are detachable and reattachable along the wrap (e.g., via hook and loop mechanisms). Thus, in some embodiments removable electrodes may be affixed (e.g., via skin-contact adhesive) to certain locations on a patient by a provider, and the compartments adjusted such that they will align with the ports when the article is worn. The ports, in turn may align with a processing device 610. The processing device 610 may include a battery, data link, filtering circuitry (e. g, a DSP or custom analog filtering circuity), memory, etc. as described above. In some embodiments, device 610 may fit within a slim or contoured housing 612, adapted to hold the device 610 in place within a compartment 604.

In some embodiments, the network environment 100 may support simultaneous use of multiple EMG devices 112 or multi-channel sensor configurations to enable bilateral or multi-site EMG acquisition. For example, a first EMG device 112 may be positioned on a patient's right lower limb and a second EMG device 112 may be positioned on the patient's left lower limb, with both devices transmitting EMG data concurrently to the computing device 105-a. The computing device 105-a may process the bilateral EMG data to compute muscle balance metrics, such as activation amplitude ratios or timing symmetry indices between contralateral limbs, enabling providers to assess whether a recovering limb has achieved activation parity with the uninjured contralateral limb. Bilateral or multi-site monitoring may be of particular relevance in athletic training and injury prevention contexts, where muscle imbalance between limbs may indicate elevated injury risk or incomplete recovery, and where return-to-sport authorization may depend on objective demonstration of bilateral activation symmetry. In some embodiments, the gamified application may incorporate bilateral EMG data into gameplay mechanics, for example by requiring coordinated activation of both limbs to control different aspects of character movement or by presenting comparative feedback displays showing activation levels for each monitored site.

In some embodiments, the computing device 105-a may compute specific bilateral activation ratio metrics, such as a limb symmetry index expressing the ratio of the recovering limb's mean or peak activation amplitude to that of the contralateral limb, and may compare the computed ratio against a provider-specified threshold (e.g., ninety percent activation parity) to generate an objective return-to-sport readiness indicator. The bilateral ratio metrics may be tracked longitudinally across multiple sessions and presented to the provider via the provider data store database 130, enabling the provider to monitor the trajectory of bilateral symmetry recovery and to make evidence-based decisions regarding return-to-sport authorization or progression of the rehabilitation protocol.

Because the EMG device 112 measures the electrical activity of the muscle directly rather than inferring muscle engagement from observed movement, the system described with respect to FIG. 2 promotes consistency in muscle activation quality rather than mere detection of gross motion. A patient performing a prescribed exercise with proper muscle engagement and a patient performing the same apparent movement through compensatory motion, momentum, or recruitment of non-target muscle groups may produce similar results on a camera-based motion tracking system but will produce distinguishable EMG activation patterns detectable by the EMG device 112. This distinction contributes to allowing the gamified exercise system 110 to provide feedback and scoring that rewards correct, sustained, and controlled muscle activation at prescribed intensity levels, making the system a more personalized and anatomically specific rehabilitation tool compared to approaches that rely solely on observation of limb position or movement trajectory.

In some embodiments, the gamified exercise system 110 may differentiate between concentric, eccentric, and isometric muscle contractions by correlating EMG activation amplitude and temporal profile data from the EMG device 112 with joint angle or limb movement data from an inertial measurement unit (IMU) or other motion sensor integrated into the wearable apparatus, and/or based on human movement data acquired through a computer vision device such as a depth camera. For example, an EMG activation pattern occurring simultaneously with joint angle change in a shortening direction may be classified as a concentric contraction, an EMG activation pattern occurring simultaneously with joint angle change in a lengthening direction may be classified as an eccentric contraction, and an EMG activation pattern occurring without appreciable joint angle change may be classified as an isometric contraction. The classified contraction type may be compared against the provider-prescribed contraction type for the current exercise, and the gamification module may provide feedback indicating whether the patient is performing the correct type of contraction, award points or progression credit only for contractions matching the prescribed type, or adjust game mechanics to encourage the prescribed contraction pattern.

FIG. 3 illustrates a signal processing and control system 300 for translating EMG signals from the EMG device 112 into game control commands according to aspects of the present disclosure. It is contemplated that the system 300 disclosed within FIG. 3 can be implemented within the network environment 100 of FIG. 1 and other examples disclosed herein. Here, the system 300 comprises a signal processing pipeline 310 that receives raw EMG data from the EMG device 112 and processes it through multiple stages to generate responsive, stable control outputs that may control movement or other interactive elements within the gamified application on a user interface. The signal processing pipeline 310 includes an RMS calculation module 312, an average calculation module 314, a PID controller constants module 316, a threshold module 318, and output states, including a spaceman moves up state 320 and a no movement state 322.

Within FIG. 3, the RMS calculation module 312 receives raw EMG signal data from the EMG device 112 and applies root mean square filtering to quantify muscle activation amplitude while reducing noise. The filtering may be performed via a processor or circuitry local to an EMG device 112 and electrically coupled and/or wired to the EMG sensor, or may be performed via a processor that is not local to the device 112 and instead receives EMG data via a data link. The RMS calculation module 312 may operate on a sliding or rolling window of raw EMG samples, computing the square root of the mean of the squared values within the window according to the formula RMS=√(1/nΣx²), where n represents the number of samples in the window and x represents individual sample values. The window size may be configurable based on the sampling rate of the EMG device 112 and desired responsiveness characteristics, with larger windows providing greater noise reduction at the cost of increased latency, and smaller windows providing faster response at the cost of potentially more signal variability. The output of the RMS calculation module 312 is a continuous signal representing the envelope or amplitude of muscle activation over time, which is then passed to subsequent processing stages.

Within the example of FIG. 3, the average calculation module 314 receives the RMS-filtered signal from module 312 and applies additional smoothing through a moving average filter to further reduce jitter and temporal variations in the signal. The moving average calculation may compute the arithmetic mean of RMS values over a specified time window, effectively low-pass filtering the signal to remove high-frequency components that could cause erratic or unstable character movements. In various examples, the average calculation module 314 may implement simple moving average, weighted moving average, or exponential moving average algorithms depending on desired smoothing characteristics and computational efficiency considerations. The smoothed output from the average calculation module 314 represents a stable measure of sustained muscle activation that accurately reflects user effort while minimizing artifact from transient electrical noise, motion artifact, or brief involuntary muscle twitches.

Still referring to FIG. 3, the PID controller constants module 316 stores and provides proportional, integral, and derivative gain values that are used to translate the smoothed EMG signal into control outputs. A proportional-integral-derivative (PID) controller implements a feedback control algorithm that continuously calculates an error value as the difference between a desired setpoint and a measured process variable, then may apply corrections based on proportional, integral, and derivative terms. In the context of the present system, the PID controller may use the smoothed EMG signal as the process variable and generates control outputs that regulate character movement velocity or position. The proportional term (Kp) produces an output proportional to the current error, providing an immediate response to changes in muscle activation. The integral term (Ki) accounts for accumulated past errors, helping to eliminate steady-state offset and ensure the character reaches the desired position. The derivative term (Kd) predicts future error based on the rate of change, providing damping to prevent overshooting and oscillation. The PID controller constants module 316 may store default gain values and also allow for adjustment or tuning of these values based on user preferences, system calibration results, or adaptive algorithms that optimize control responsiveness for individual users.

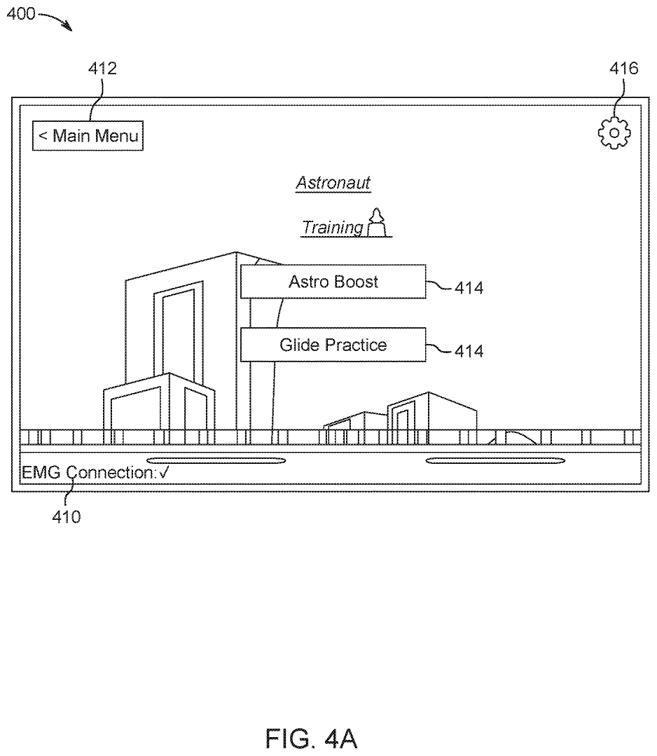

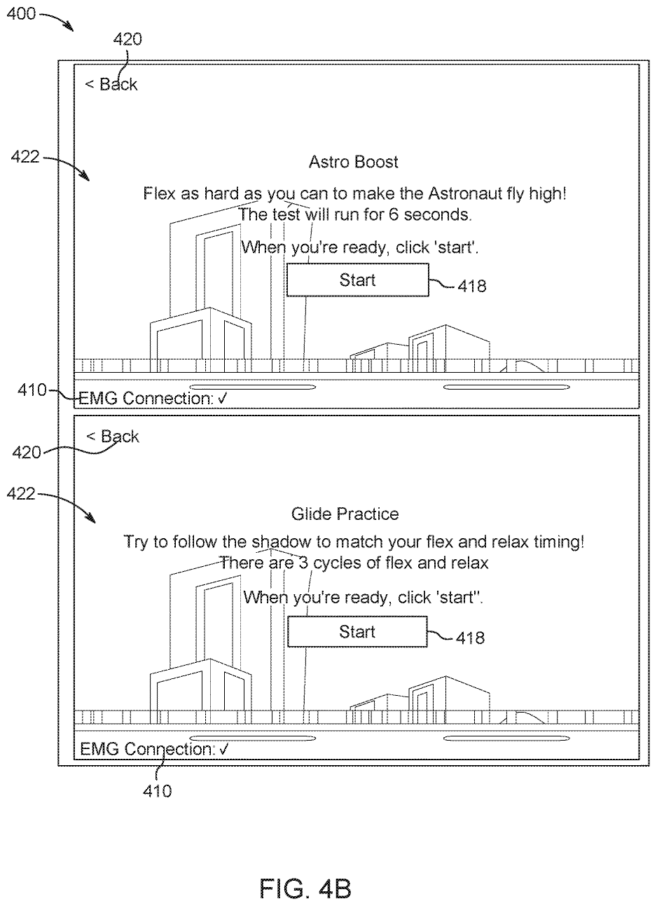

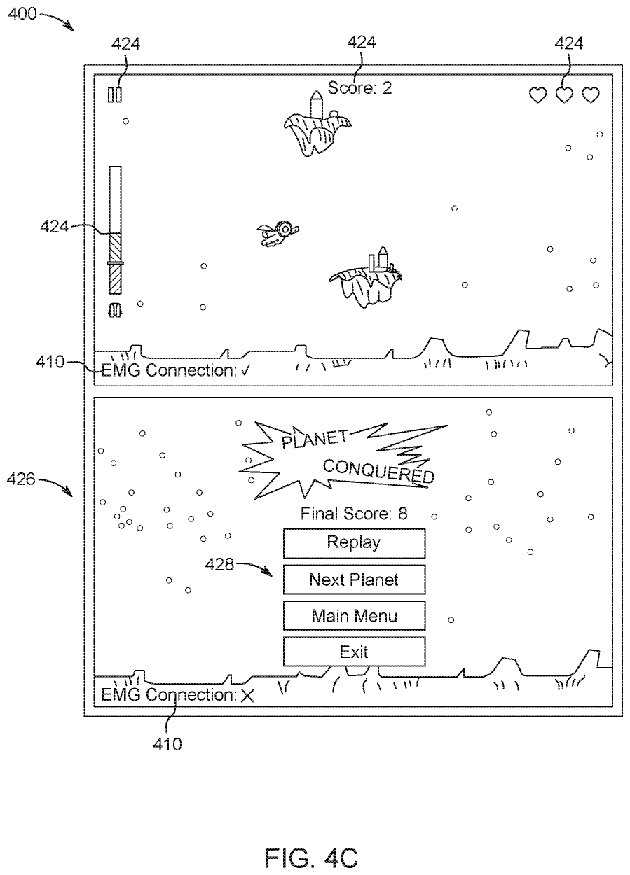

In some embodiments, the proportional, integral, and derivative gain values (Kp, Ki, Kd) stored in the PID controller constants module 316 may be individually determined for each user through the Glide Practice calibration protocol described with respect to FIG. 4B. During the Glide Practice protocol, the user is prompted to follow a guiding character by contracting and relaxing the target muscle in a controlled, cyclical manner. The system records the user's EMG activation profile during these guided contraction-relaxation cycles and derives personalized PID gain values based on the user's demonstrated response characteristics, including the speed at which the user can initiate and terminate muscle activation, the degree of overshoot or undershoot relative to the target activation level, and the stability of sustained contractions. Users who demonstrate rapid, precise control may receive gain values that preserve high responsiveness, while users who demonstrate slower or more variable control may receive gain values that provide greater smoothing and damping to facilitate successful gameplay.

In some embodiments, the signal processing pipeline 310 may compute a per-user sensitivity factor to normalize game control responsiveness across users with differing natural EMG signal strengths. The sensitivity factor may be derived during the calibration protocol by collecting rate-of-change (ROC) values of the smoothed RMS signal, sorting the collected ROC values, and identifying a statistical percentile value (e.g., the ninety-ninth percentile) to establish a reference magnitude that excludes outlier spikes. The sensitivity factor may then be computed as a normalization coefficient based on the identified percentile value, such that users with naturally stronger EMG signals and users with naturally weaker EMG signals produce comparable game control outputs when exerting proportionally similar effort. Application of the per-user sensitivity factor ensures that gameplay difficulty and responsiveness are calibrated to each individual's neuromuscular characteristics rather than being dependent on absolute EMG amplitude, which may vary significantly across patients due to differences in subcutaneous tissue thickness, electrode placement, muscle fiber composition, or disease state.

Further illustrated within FIG. 3, the threshold module 318 receives the processed control signal and compares it against one or more threshold values to determine the appropriate output state. The threshold module 318 may implement decision logic that translates continuous muscle activation levels into discrete game states or actions. In the example shown, the threshold module 318 evaluates whether the processed EMG signal exceeds a predetermined activation threshold, and based on this comparison, selects between the spaceman moves up state 320 or the no movement state 322. The threshold value used for comparison may be derived from personalized calibration data, such as a specified percentage of the user's maximum voluntary contraction amplitude determined during the calibration protocols described elsewhere herein. In various examples, the threshold module 318 may implement hysteresis or dead-zone logic to prevent rapid state transitions when the signal hovers near the threshold value, providing more stable and predictable character behavior.

When the processed EMG signal exceeds the threshold, the system enters the spaceman moves up state 320, wherein the game character (such as an astronaut, spaceman avatar, or other configurable character) ascends vertically within the game environment. The rate or velocity of upward movement may be proportional to the magnitude by which the EMG signal exceeds the threshold, providing continuous feedback where stronger muscle activation produces faster character movement. Alternatively, the upward movement may occur at a fixed velocity whenever the threshold is exceeded, providing binary on/off control that may be easier for some users to master. The spaceman moves up state 320 continues as long as the EMG signal remains above the threshold, allowing users to sustain character elevation through maintained muscle contraction.

In some embodiments, the rate or velocity of character movement in the spaceman moves up state 320 may be determined based on the rate of change (ROC) of the smoothed RMS signal rather than, or in addition to, the absolute magnitude of the signal. The system may compute the ROC by determining the difference between the current smoothed RMS value and the immediately preceding smoothed RMS value, and may apply a dampening function, such as a square root transformation, to the computed ROC to attenuate the effect of sudden signal spikes while preserving proportional responsiveness to sustained changes in activation. The dampened ROC value may then be scaled by the per-user sensitivity factor and a tuning constant (scaling factor) to produce a movement speed value. The movement speed value may further be capped at a maximum boost value to prevent the character from exceeding a predetermined maximum velocity regardless of signal magnitude, thereby maintaining playability and preventing the character from moving off-screen during extreme activations. The resulting movement speed formula may be expressed as: movement speed=min(√(ΔRMS)×sensitivity×scaling factor, max boost), where ΔRMS represents the change in the smoothed RMS value between successive frames.

When the processed EMG signal falls below the threshold, the system enters the no movement state 322, wherein the game character ceases upward movement and may descend, hover, or fall according to game physics rules as programmed. In various examples, the no movement state 322 may cause the character to descend at a constant velocity simulating gravity, gradually decelerate, and come to rest at a neutral position, or maintain its current vertical position until the next activation. The transition between the spaceman moves up state 320 and the no movement state 322 creates a direct, intuitive mapping between muscle activation and character behavior, allowing users to control vertical position through voluntary muscle contraction and relaxation cycles.

The signal processing pipeline 310 may operate continuously in real-time during gameplay, processing a stream or readout of incoming EMG signals (or a sampling thereof) at rates sufficient to provide responsive feedback with minimal perceptible latency between muscle activation and visual response. In various examples, the pipeline 310 may process data at frame rates synchronized with the game rendering engine (e.g., 30, 60, or 120 Hz) or at higher rates determined by the EMG device's measurement data readout sampling frequency. The modular architecture of the pipeline 310 allows individual processing stages to be enabled, disabled, or reconfigured based on user needs, device capabilities, or therapeutic protocols. For example, the degree of smoothing applied by modules 312 and 314 may be adjusted based on calibration results indicating user control capability, with users demonstrating smooth, controlled activations receiving less filtering to preserve responsiveness, while users with more variable or tremulous signals receive more aggressive smoothing to facilitate successful gameplay.

The signal processing pipeline 310 may further include an error detection module configured to monitor the communication link between the EMG device 112 and the computing device 105-a during operation. The error detection module may actively poll or monitor the connection status at critical junctures, including when the user initiates gameplay or calibration, during active data collection, and at periodic intervals throughout a session. If the EMG device 112 becomes disconnected or ceases to transmit valid data, the error detection module may cause the computing device 105-a to display an on-screen error message prompting the user to verify the hardware connection, and may safely halt gameplay execution to prevent data corruption, misleading biofeedback, or scoring based on absent or invalid EMG input.

The EMG connection indicator 410 described with respect to FIGS. 4A-4C may be updated in real time to reflect the current connection status, transitioning from a checkmark symbol to an X symbol upon detection of a disconnection event. Additionally, FIG. 4B includes another example of interfaces 400 for use with the gamified exercise system 110. Again, the EMG connection indicator 410 is shown, illustrating proper connection with the hardware device via a checkmark symbol. Start buttons 418 are shown on the user interface, which the patient or provider may use to begin the game or test after reviewing the instructions. Shown on the interface 400, a back button 420 may also be included to proceed back to the previous screen, allowing users to navigate between different sections of the application without losing progress or requiring an application restart. Each interface 400 may also include an instructional note 422 providing instructions for a patient or provider, such as “Flex as hard as you can to make the Astronaut fly high! The test will run for 6 seconds” for the Astro Boost calibration, or “Try to follow the shadow to match your flex and relax timing! There are 3 cycles of flex and relax” for the Glide Practice calibration. These instructional notes 422 may be customized and appropriate for pediatric users while conveying the specific task requirements and duration, ensuring users understand what is expected before initiating each calibration protocol.

FIG. 4c includes additional interface 400 examples for use with the gamified exercise system 110 during active gameplay and upon level completion. The upper portion of FIG. 4c shows an active gameplay screen where an astronaut avatar navigates through a space environment containing floating island obstacles, with the character's vertical position controlled by EMG activation as processed through the signal processing pipeline of FIG. 3. Here, the interfaces 400 include an example EMG connection indicator 410 showing both paired (checkmark) and unpaired (X) connection states in different screens. FIG. 4c also includes a plurality of scoring icons 424 to assist in feedback display and game enhancement, including a vertical bar graph or “jet boost” indicator 424 on the left side of the screen displaying real-time EMG activation level, a numerical score display 424 at the top center showing accumulated points, and heart icons 424 in the upper right corner representing remaining lives in the multi-life system. The lower portion of FIG. 4c shows a level completion screen displaying “PLANET CONQUERED” with a final score and presenting buttons 428 for various game control options, including “Replay” to repeat the current level, “Next Planet” to advance to the subsequent level in the progressive difficulty structure, “Main Menu” to return to the primary navigation screen, and “Exit” to close the application. The completion screen 426 provides positive reinforcement through celebratory graphics and messaging, supporting sustained motivation and engagement throughout the therapeutic protocol.

The illustrated interfaces 400 and various data collected from gameplay may be stored or transferred to at least one provider data store database 130 or other elements of the gamified exercise system 110 as described herein. For example, during sessions, the system continuously records timestamped raw EMG signal values at the native sampling rate of the EMG device 112, processed signal values including RMS amplitudes and smoothed activation levels output from modules 312 and 314, calibration results from the Astro Boost and Glide Practice protocols, including maximum voluntary contraction values and smoothness metrics, gameplay events such as level initiation, completion, and failure instances, character position data, threshold crossings, and total session duration.

In some embodiments, the system may automatically export session data in one or more standardized file formats to facilitate provider review and longitudinal analysis. For example, raw and processed EMG signal data from each session may be saved as comma-separated value (CSV) files containing timestamped signal values, and corresponding signal visualizations may be automatically generated and saved as image files (e.g., PNG format) depicting the raw EMG signal waveform, the RMS-filtered signal, and annotations indicating maximum activation values and threshold levels. The exported files may be organized and stored in a directory structure associated with the user's profile, enabling systematic retrieval and comparison of data across multiple sessions. The exported data files may also be transmitted to the provider data store database 130 for remote access by authorized providers.

Upon completion of a level or session, the logged data may be compiled into session summary records that include aggregate statistics such as average activation levels, peak activation values, number of successful threshold crossings, adherence metrics indicating completion of prescribed repetitions or duration targets, and scores 424 displayed through the user interface 400. The compiled data may be stored locally on the computing device executing the application and simultaneously transmitted via network connection to the provider data store database 130 described in relation to FIG. 1, where it becomes accessible to authorized healthcare providers through remote monitoring interfaces. The data transmission may occur in real-time during gameplay, at the conclusion of each session, or according to a scheduled synchronization protocol depending on network availability and system configuration. In various embodiments, the system may implement data encryption during transmission and storage to protect patient health information in compliance with applicable privacy regulations, such as HIPAA, and may include data integrity checks, such as checksums or digital signatures, to ensure that transmitted records have not been corrupted or tampered with during transfer from the patient device to the clinical data store.

### Example Methods and Processes

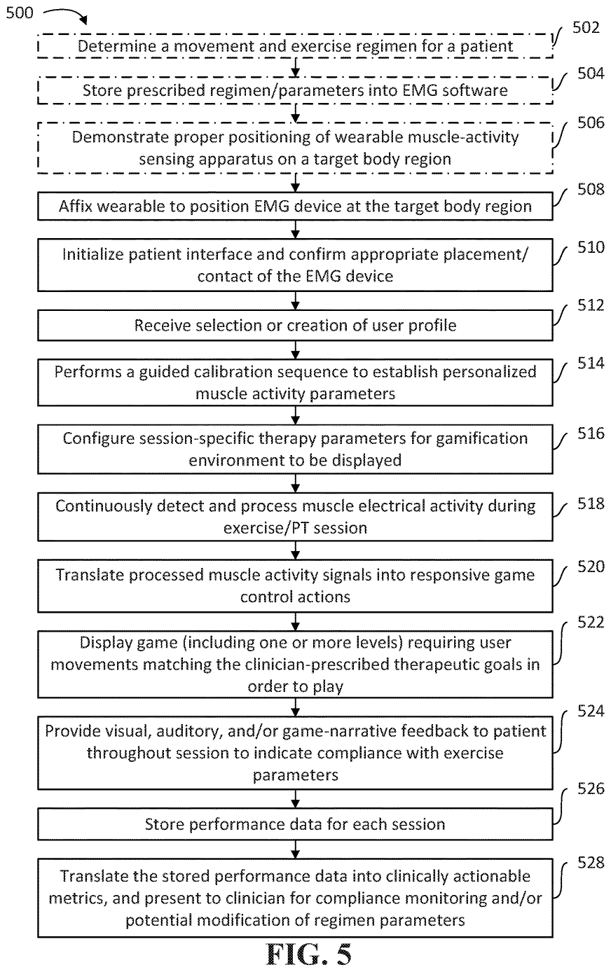

FIG. 5 illustrates an example method 500 (or set of interacting methods) by which the features and attributes described herein can be employed to promote patient compliance, adherence, and consistent accuracy in performing prescribed physical therapy or training regimens. Example method 500 includes a variety of actions or steps from initial set up and prescription through a patient's use of a gamified physical therapy rehabilitation system in accordance with one or more aspects of the present disclosure. Some or all of these steps may be parsed into separate methods performed at or by a physical therapist or healthcare provider, by the patient, or by a networked software system connecting to specific equipment.

The method 500 may be performed using one or more components of the network/system 100 described with respect to FIG. 1, including the gamified exercise system 110, the EMG device 112, the computing devices 105, and/or the provider data store database 130. It should be understood that the blocks of the method 500 described below need not be performed in the exact order depicted, and that one or more blocks may be omitted, repeated, reordered, separated, or combined in various embodiments, depending on the therapeutic context, deployment environment, and clinical workflow. Each block is described below with reference to the corresponding element of FIG. 5.

At block 502, the method 500 optionally includes determining a movement and exercise regimen for a patient. In some embodiments, a clinician, physical therapist, athletic trainer, or other authorized healthcare provider examines the patient and determines a movement and exercise regimen to be prescribed to the patient. The regimen may specify one or more target muscles to be engaged during therapeutic exercises, which may include an injured or affected muscle itself, surrounding or protective muscles (e.g., muscles supporting a joint following ligament reconstruction), or both. The regimen may further specify one or more parameters associated with engagement of each target muscle, including, but not limited to, target activation intensity level, contraction type or exertion pattern (e.g., concentric, eccentric, or isometric), contraction duration, number of repetitions and sets, sequencing of movements across multiple muscle groups, rest intervals, session frequency, and acceptable tolerance ranges defining upper and lower bounds within which the patient's activation should fall before the system adjusts difficulty or provides corrective feedback. In some embodiments, the provider may specify these parameters using clinical rehabilitation terminology familiar to the provider, and the system may translate such clinical parameters into corresponding gameplay parameters as described with respect to the clinic configuration module herein. In other embodiments, a software network may simply extract this information from an EMR, a scan of a PT's take-home sheet, or use predefined regimens for given therapeutic needs of a patient.

At block 504, the method optionally includes storing the regimen parameters into EMG software. For example, in some embodiments a provider enters the prescribed regimen parameters into the system. The parameters may be entered via any of several input modalities, including entry into an electronic medical record in standard clinical parlance, entry into a prescriptive narrative document associated with the patient's care plan, entry via a provider-facing user interface associated with the clinic configuration module on the computing device 105-b using clinical terminology that the system subsequently translates into gameplay parameters, or direct specification of gameplay settings such as activation thresholds, obstacle configurations, level structure, and session timing. The entered parameters may be stored in a provider data store 130 and made accessible to the computing device 105-a for retrieval at the start of a subsequent therapy session.

At block 506, which may be optional in some embodiments, the provider assists the patient with assembling and fitting a wearable muscle-activity sensing apparatus on the target body region. This may include a belt, strap, harness, girdle, sleeve, shirt, brace, or similar article that can be worn so as to affix a sensor securely on a patient's body while they exercise. The provider may demonstrate to the patient the correct placement of the EMG device 112, including positioning of surface electrodes, IMU sensors, or other sensing components which are attached or will attach to the wearable article, and may verify that adequate skin contact and sensor alignment have been achieved. The wearable sleeve, strap, or garment may be sized and configured to be tailored to the patient's morphology such that the form factor of the garment encourages or constrains sensor placement to anatomically correct positions when worn, thereby promoting accurate and repeatable measurements across sessions. Block 506 may be performed during an initial clinical visit and omitted during subsequent at-home sessions once the patient has demonstrated proficiency in self-application of the device. Alternatively, block 506 may be performed via video or animation shown to the patient during their own use when performing (or first performing, or on demand) their exercises.

At block 508, the patient puts on the wearable muscle-activity sensing apparatus (e.g., by affixing hook and loop straps, putting on a shirt or sleeve, strapping on a knee brace, etc.) and adjusting the wearable portion so as to position the attached EMG sensors on the target body region in accordance with the placement guidance provided by the labels 212 or other visual indicators described with respect to FIG. 2, below. In some embodiments, a user may first put on the wearable sleeve/article, and then subsequently affix the sensor via grommets or other connection points so as to ensure they are touching the correct portion of the patient's body over a muscle of interest.

At block 510, the method 500 may confirm appropriate placement of the wearable apparatus, which may include determining whether sufficient skin contact is made so as to provide suitable EMG sensing data and/or determining whether the right muscle is being sensed. Confirmation may be performed by instructing the patient to perform one or more test movements and monitoring the resulting EMG signals to determine whether they fall within a plausible range expected for proper sensor positioning, by utilizing optical cameras or depth sensors to verify device orientation relative to anatomical landmarks, or by presenting on-screen visualizations or guidance prompts directing the patient to adjust the device until signal quality metrics meet a predetermined threshold. In some embodiments, supplemental sensors such as an inertial measurement unit (IMU) integrated into the wearable apparatus may provide orientation data indicating whether the patient is standing, sitting, or lying down, which the system may use to contextualize the expected EMG signal characteristics and further validate proper device placement.

At block 512, the patient or caregiver activates the therapeutic system to begin exercises. The patient may pair the wearable muscle-activity sensing apparatus with the game interface on the computing device 105-a, if not already done. The computing device 105-a may establish a wireless communication link with the EMG device 112 via Bluetooth, Bluetooth Low Energy, Wi-Fi, or other suitable wireless protocol, and the user interface may display a connection status indicator (e.g., the EMG connection indicator 410 described with respect to FIGS. 4a-4c) confirming successful pairing before proceeding to subsequent blocks. The computing device may then display a user interface and game environment on a display screen, allowing the patient to select or create a user profile on the computing device 105-a. Through this profile, the system loads any previously established session parameters, calibration data, and performance history associated with that profile. The session parameters may be retrieved from local storage on the computing device 105-a, from the provider data store database 130 via a network connection, or from both sources. Loading of prior session data enables the system to resume therapy at the appropriate difficulty level and to apply previously determined calibration values without requiring the patient to repeat the full calibration sequence at every session.

The user profile management functionality on the computing device 105-a may include interfaces for creating new user profiles, selecting from previously created profiles, and switching between profiles during a session. When creating a new profile, the user or caregiver may enter identifying information such as a name or identifier, and the system may initialize a new data record for storing calibration data, session history, and performance metrics associated with that profile. When returning to the system, the user may select an existing profile from a list of previously created profiles, causing the system to load the associated calibration data, session parameters, and performance history. A ‘change user’ function may allow a different user to switch to their own profile without restarting the application, facilitating multi-user deployment in clinical or household settings where multiple patients may share a single computing device and EMG device.

At block 514, the method 500 performs a guided calibration sequence to establish personalized muscle activity parameters governing gameplay control and challenge levels. The calibration sequence may include a maximum voluntary contraction assessment (e.g., the Astro Boost protocol described with respect to FIG. 4b) to determine the patient's peak EMG activation amplitude, and a control smoothness assessment (e.g., the Glide Practice protocol described with respect to FIG. 4b) to evaluate the patient's ability to modulate muscle activation in a controlled manner. The resulting calibration data, including maximum activation values, smoothness metrics, and derived handicap coefficients, may be stored in the user profile for application during gameplay. In some embodiments, block 514 may be performed at every session; in other embodiments, block 514 may be performed only at the initial session or periodically thereafter, with previously stored calibration data applied at intervening sessions.

At block 516, the system configures session-specific therapy parameters, including challenge level, activation targets, repetition goals, and session duration, based on the provider-prescribed regimen retrieved at block 504 and the calibration data established at block 514. The gamification module may apply the configured parameters to set obstacle configurations, threshold values, level progression requirements, and scoring criteria for the upcoming session. In some embodiments, the patient may also select a game type, avatar, visual theme, or other gameplay preferences at this block.

At block 518, the system continuously detects and processes muscle electrical activity during the therapeutic session. The EMG device 112 acquires raw EMG signals from the target muscle group and transmits the signals to the computing device 105-a, where the signal processing pipeline 310 described with respect to FIG. 3 applies RMS filtering, moving average smoothing, and PID control calculations to generate stable, responsive control outputs. The system monitors the processed signals relative to the patient's established baseline to detect changes indicative of muscle engagement, strain, fatigue, or relaxation.

At block 520, the system translates the processed muscle activity signals into responsive game control actions. The processed EMG signals may be mapped to generic game control parameters such as velocity, direction, acceleration, or force, which the gamification module then applies as though they were commands received from a conventional game controller. For example, muscle activation exceeding a threshold may cause a game character to ascend, accelerate, or interact with environmental elements, while relaxation below the threshold may cause the character to descend, decelerate, or remain stationary, as described with respect to the spaceman moves up state 320 and no movement state 322 of FIG. 3.

At block 522, the system presents the user with a game (e.g., an ‘app’-style game or video game) the rules and gameplay of which will influence, cause, or require the user to perform the prescribed exercise or PT movements in order to score, advance, or win in the game. The method may display sequential game levels containing game elements that require user movements matching the provider-prescribed therapeutic goals, with each level including one or more sets of one or more exercise motions. One or multiple levels may comprise game elements that require performance of the movements of the whole regimen. The gamification module renders obstacle configurations, passage zones, and environmental elements whose spatial and temporal properties correspond to the therapeutic parameters configured at block 516, such that successful navigation of the game environment requires the patient to perform muscle activations of the prescribed intensity, duration, and frequency. The system may advance the user to progressively challenging exercise targets, unlocking higher levels as the user demonstrates adequate performance at each preceding level.

At block 524, the system provides visual, auditory, and game-narrative feedback throughout the session to reinforce user effort and maintain engagement. Visual feedback may include real-time EMG activation level indicators (e.g., the jet boost bar described with respect to FIG. 4c), scoring displays, animated effects responsive to threshold crossings, and celebratory graphics upon level completion. Auditory feedback may include background music, sound effects triggered by gameplay events, and auditory cues indicating successful or unsuccessful performance. Game-narrative feedback may include progression through themed environments, unlocking of new content, and positive reinforcement messaging designed to sustain motivation across extended rehabilitation periods.

At block 526, upon session completion or at defined intervals during the session, the system stores performance data, including gameplay metrics and associated EMG signal records and muscle activity measurements, in association with the user profile. The stored data may include game (session)-level movement and muscle engagement-related information, like timestamped raw and processed EMG values, threshold crossing events, number of reps performed, level completion status, muscle engagement duration, scores, and calibration data. The stored data may also include metadata regarding the session itself, like time of day, day of week, date, number of pauses or interruptions, etc. And, the stored data may further include higher order information, like trends or aggregate data on overall compliance with regimen exercise parameters (duration, intensity, etc.), adherence data such as frequency of using the game and performing the exercises of the regimen, user comments or feedback, and any data that might indicate methodical non-compliance or non-effort (e.g., the user is always laying down when doing the sessions, or the session to session EMG values indicate different people are using the system). The data may be stored locally on the computing device 105-a and transmitted to the provider data store database 130 for remote access by authorized providers, as described with respect to FIG. 1.

At block 528, the system translates the stored performance data into clinically actionable metrics, including compliance indicators (e.g., whether the patient completed the prescribed regimen, session timing, duration, and proportion of prescribed repetitions completed), adherence trend data (e.g., changes in session frequency or engagement duration over time that may indicate declining motivation), specific movement and activation performance metrics (e.g., peak and average activation amplitudes, activation consistency, and fatigue progression), and other summary information accessible to the provider via the computing device 105-b or the provider data store database 130. Based on review of these metrics, the provider may adjust future session parameters, including modifying activation thresholds, repetition targets, difficulty progression rates, or therapeutic modes, via the clinic configuration module. In some embodiments, the system may automatically suggest or implement parameter adjustments based on predefined rules or adaptive algorithms without requiring explicit provider intervention between sessions.

### Example Implementations, Validation Studies, and Experiments

The inventors conducted several studies and experiments validating the approaches described herein. These validation studies were conducted to demonstrate and quantify the advantages of the overall scope of embodiments contemplated. Although the studies were performed using certain implementations and configurations, it should be understood that the results of these studies establish the advantages of all contemplated embodiments. In other words, the particular examples used for purposes of the validation studies are not intended as limiting. One such experiment is described below, to serve as an illustrative example of the improvements achieved by embodiments of the present disclosure versus previous approaches, in terms of patient engagement and sustained participation in therapeutic exercises, responsiveness and accuracy of muscle-activity-driven game control, adaptability of therapy parameters to individual patient capabilities, and the quality and clinical utility of longitudinal performance data captured during rehabilitation sessions.

### 1. Introduction of Validation Embodiment Development

Pediatric physical disability, particularly those affecting mobility, is a significant public health concern, with thousands of children experiencing impairment due to neuromuscular or traumatic causes. According to recent epidemiological data, lower limb conditions (e.g., fractures, cerebral palsy, muscular dystrophies) are among the most common causes of physical disability in adolescents. These conditions frequently result in reduced muscle strength, limited joint mobility, and impaired balance/coordination, often requiring long-term rehabilitation to restore function. Sports-related lower extremity injuries account for a large proportion of cases, with a high prevalence of ankle and knee injuries. For those with developmental or neuromuscular disorders, mobility limitations often persist into adulthood, further underscoring the need for early and effective intervention strategies.

Subsequently, engagement in physical rehabilitation is a key determinant of success in restoring or maintaining functional mobility, particularly in pediatric populations facing challenges related to lower limb strength and coordination. Tasks such as ambulating, rising from a seated position, and navigating stairs are foundational to independent living, yet they often present significant hurdles for children undergoing rehabilitation due to neuromuscular or orthopedic conditions. Traditional therapy approaches, while clinically effective, may fall short in maintaining sustained participation, especially in younger patients who benefit from more interactive and motivating therapeutic environments.

To address this challenge, the present disclosure proposes the development of a gamified rehabilitation system, Galactic Glide, designed to enhance participation and improve outcomes for lower leg mobility exercises in adolescent populations. The system integrates a wearable sensor array capable of capturing electromyography (EMG) signals with an engaging computer-based video game. Through real-time signal collection and feedback, children are able to visualize movements as game actions, reinforcing physical therapy in a way that is both intuitive and enjoyable. Additionally, the system incorporates adaptive personalization that allows for clinician-guided tuning and progression tracking. By aligning therapeutic goals with engaging gamification, the platform aims to sustain long-term motivation and improve functional outcomes of physical therapy for adolescent populations.

### 2 Application of Gamification Principles

Gamification is the use of game design elements alongside mechanics in non-game contexts to increase engagement, motivation, fun, and passion across tasks. The inventors have found that this can be a particularly powerful aid in the context of physical therapy for children and adolescents. This demographic often shows low rates of PT regimen compliance and long-term adherence, which can be quite detrimental during stages of growth that occur during childhood and adolescence. Thus, there is a particular need to promote adherence to PT regimens for these individuals.

At the same time, this population can have more advanced expectations for their entertainment and attention. Merely gamifying a PT routine in a general sense (e.g., instructing an adolescent patient to “see how many you can do in 5 minutes”) has been found by the inventors not to be sufficiently motivating or engaging. Based on the inventors' review, evidence suggests that children expect intelligent user interfaces (IUIs) to be customized and tailored to user preferences, and that IUIs should include visual screen elements, including animations and effects.

Moreover, the inventors have found that a helpful design attribute is to ensure that the gamification features being utilized correlate more directly to the specific behavior or movements that must be performed. In the PT context, specific physical motions and specific muscle engagements are often prescribed as part of the PT regimen and so these specific movements/engagements should be encouraged, guided, and tracked through the gamification. In contrast, video game environments that merely promote general goals like “more movement” or even sequences of gross motion would not be correlated to a specific muscle engagement, training, and/or motion in a suitably specific way to serve the goals of physical therapy. For example, methods that rely on generalized or widely available video game equipment (Nintendo Switch, Nintendo Wii, X-Box Kinect, etc.), which do not incorporate EMG sensing or other detection of specific muscle engagement and strain, were not found to be suitable for PT. For example, they would not allow for specific muscle measurements, feedback to the patient on degree of strain/motion, or for personalized therapy progression.

### 3 System Design

### 3.1 Wearable Design

The wearable was designed to be lightweight, form-fitting, secure, and compatible with EMG-based signal acquisition. In the inventors' validation embodiments, a number of particular electronic and wearable/sleeve components were utilized, which are consistent with some or all of the more general descriptions above. For purposes of this discussion, FIG. 6 can be considered as a conceptualization of the inventors' validation embodiment, depicting a wearable system hardware assembly, having connectivity, sensors, processing, a housing, etc. FIG. 2 illustrates a sleeve worn on a limb such that an EMG sensor is positioned on a leg muscle of interest, also conceptually consistent with the validation experiments.

The inventors' validation embodiment included a custom-fabricated wearable double-layered neoprene sleeve (such as shown in FIG. 2) with a fitted compartment to securely house a MyoWare 2.0 Wireless Shield and Muscle Sensor (such as shown in FIG. 2 and FIG. 6). The sleeve comprised a lining of unbroken loop fabric for adjustable Velcro attachment. Further, it was configured for use on either leg, where upward-facing right/left arrows allowed for corresponding limb use. This layered design was formulated to ensure comfort, modularity, and stable sensor positioning throughout gameplay.

For electronics, the validation embodiment made use of several dev-kit components. Primarily, the validation embodiment utilized a MyoWare 2.0 Muscle Sensor (e.g., as conceptually depicted in FIG. 6) which recorded superficial EMG activity from the user's lower leg via three disposable gel electrodes, two for signal detection and one for reference. These surface electrodes adhered to the skin in a standard bipolar electrode configuration on the calf (e.g., as shown in FIG. 2). The sensor snapped directly into the MyoWare 2.0 Wireless Shield (e.g., as depicted in FIG. 6), which transmitted EMG data wirelessly to an ESP32-WROOM microcontroller (e.g., processor 150 as described with respect to FIG. 1) that was serially connected to a PC running Galactic Glide (e.g., computing device 105 as described with respect to FIG. 1). Custom housings were fabricated for consumer dev-kit deployment (e.g., as conceptually depicted in FIG. 6). These custom housings were made to accommodate electronics while maintaining sensor alignment, in addition to embedding clips to prevent device slippage during movement.

The modularity of the validation embodiment's design allowed easy access for electronic removal and recharging between sessions. The snug fit ensured good electrode contact and minimized motion artifact, which is important for clean signal acquisition. In general, this design enabled the safe and effective acquisition of EMG signals in a physically secure form factor that supported dynamic use during gameplay.

### 3.2 Application Design

Data Processing. In the validation embodiment, Galactic Glide was developed in Python, which integrated live EMG data to control gameplay via PyGame (FIG. 3). To convert raw EMG signals into input, a two-stage signal processing pipeline was used. First, root mean square (RMS) (Eq. 1) was applied to a rolling window of raw EMG values.

$$RMS = √ ( 1 / n {Σ}_{i} {x}_{i}^{2} ) \qquad Equation 1$$

This reduced noise and provided a stable measure of muscle activation over time. Subsequently, further smoothing was performed using a moving average filter to reduce jitter caused by background EMG activity. The result was a continuous signal that reflected user efforts while minimizing artifact and variability.

To translate this smoothed signal into responsive in-game motion, the validation embodiment employed a proportional-integral-derivative (PID) controller (Eq. 2). This continuously adjusted output to help regulate the astronaut's movement in response to EMG activity (e.g., strong/fast muscle activation produced faster movements, with reduced overshooting/instability).

$$u ( t ) = {K}_{p} e ( t ) + {K}_{i} {\int }_{0}^{t} e ( τ ) d τ + K\_d de ( t ) / dt \qquad Equation 2$$

Gameplay Mechanics and Engagement. Space was chosen as the context for the game because it is an engaging theme for children. According to a 2019 survey conducted in the US, UK, and China, 86% of children aged 8 to 12 are interested in space exploration. Thus, users started by completing calibration (e.g., ‘Astronaut Training’) to personalize character response (FIGS. 4A and 4B). This calibration collected data regarding maximum EMG activation (e.g., via ‘Astro Boost’) and control (e.g., via ‘Glide Practice’) for use in smoothing and personalized thresholds, ensuring fair and consistent gameplay.

Once calibrated, players engaged with the interactive gamification module, which included themed levels of increasing difficulty (FIG. 4C). During gameplay, users controlled an astronaut navigating space-themed environments. A vertical “jet boost” bar was shown as part of interactive gameplay, which offered immediate visual feedback of EMG activation. The horizontal bar in the “jet boost” pane showed the threshold to cause character ascension. These features encouraged user engagement, while also building a sense of understanding during play.

The game included five progressively challenging levels (e.g., shown in ‘Planet Select’ and ‘Level Themes’ sections of FIG. 4C), each represented by a different planet. These levels were progressively unlocked (i.e., users had to complete one to access the next) to instill a clear sense of progression and motivate continued engagement. As players advanced, they were tasked with increasing EMG activation according to calibrated thresholds. To maintain motivation and reduce discouragement, the game featured a multi-life system. Lives reset after the completion of each level, allowing users ample opportunity to complete protocols. Further, distinct level themes, sound cues, and responsive feedback made each session engaging, helping players stay consistent with their therapy routines.

### 4 Discussion

Galactic-Glide was designed with clinical integration as a core goal, addressing key factors for long-term rehabilitation success in adolescents. First, it promoted sustained engagement in repetitive therapeutic exercises by embedding them in an interactive, progressively challenging game, helping maintain motivation, often a barrier in traditional therapy. Second, it personalized gameplay based on user capability through adaptive calibration (FIGS. 4A and 4B), aligning therapeutic outcomes with user-specific EMG activation to ensure appropriate challenge and prevent discouragement. Third, it included clinician-configurable thresholds, allowing adjustable muscle activation targets, repetition goals, and session durations tailored for individuals. This flexibility supported personalized therapy progression. Fourth, robust data logging captured user engagement, progress, and EMG trends, offering clinicians actionable insights for evaluating adherence and outcomes. These features also enhanced interdisciplinary communication and supported both home and clinic-based accountability. Altogether, the Galactic-Glide validation embodiment offered a scalable, adaptable solution for improving the accessibility, consistency, and effectiveness of pediatric lower-limb rehabilitation in real-world settings.

While the design of the initial validation prototype (“Galactic-Glide”) included several features to enhance rehabilitation engagement and clinical outcomes in adolescents, further features and additional functionality are contemplated. For example, sleeves, wraps, braces, and other form factors are contemplated so as to be more tailored to specific target body regions. E.g., a knee sleeve (with patella opening for alignment) may include pockets and associated sensor ports/grommets for monitoring multiple muscle engagements about a knee; foot/ankle sleeves or elbow sleeves may likewise monitor muscle engagement for the complex musculature about those joints. Shirts and/or wraps conformed to torso geometry are contemplated for back, shoulder, and/or abdominal muscle monitoring. Additionally, EMG sensors may be integrated into these wearable items, rather than using “devkit” style EMG devices as in the validation studies (in which case the sensor, PCB, battery, processor, memory, communication link, etc. may be miniaturized and conformed to fit in a thin or flexible housing that reduces size and decreases chances of interference with movement.

The development of the “Galactic-Glide” embodiment demonstrates the advantages of the systems, devices, and methods herein (achieved, in part, through combining the described wearable biosensing features with the described gamified interfaces) to improve pediatric rehabilitation (and, for similar reasons, other rehabilitation, physical therapy, athletic training, etc.). By translating lower-limb EMG signals into meaningful, interactive gameplay, the validation system not only facilitates engagement in repetitive therapeutic exercises but also introduces a scalable framework for adaptive, clinician-informed therapy. This approach supports both real-time performance feedback and long-term monitoring, aligning with current needs in personalized digital health.

### Further Embodiments, Implementations, and Applications

The foregoing descriptions of example methods, hardware configurations, and validation studies serve both as written description of their particular subject matter and contemplated embodiments as well as more general enablement of underlying operational principles, functionality, techniques, improvements, and other features. To demonstrate the generalizability of the content of the present disclosure to a variety of practical applications, the present disclosure will now describe several additional embodiments and implementations in which some or all of such features may be leveraged to achieve new, improved, tangible, and useful results in particular fields of application.

In one implementation, a gamified rehabilitation system is embodied as a consumer-facing at-home exercise and training kit targeting a broader patient population, including adults experiencing nagging injuries from muscle imbalances, recovering from lower-extremity injuries (e.g., ACL repair, ankle reconstruction, or post-operative knee recovery) and individuals with chronic neuromuscular conditions. A kit may contain a pre-configured wearable EMG sensor sleeve, disposable electrodes, a wireless transmitter module, a charging cable, and a downloadable or pre-installed game and user interface application. The application may include an onboarding and calibration module completable without a provider present, allowing patients to self-enroll and establish personalized muscle activation thresholds from home. A companion provider-facing web portal may also be present which could allow an on-demand trainer or physical therapist to remotely configure session parameters, including target activation thresholds, number of levels or repetitions, and session duration and to review session EMG data, enabling remote adjustment of therapy parameters between sessions.

In another implementation, a system may be configured as a clinical-grade rehabilitation platform for physical therapy clinics, rehabilitation centers, hospitals, nursing homes, elder care and assisted living facilities, and sports medicine practices. The clinical version may include a dual-interface design: a provider-facing dashboard and a patient-facing game display. The provider interface may allow a therapist to actively modify therapy parameters, such as activation thresholds, difficulty levels, and exercise targets, in real time during a session. Session data may be automatically stored and associated with each patient's profile, enabling longitudinal tracking of EMG trends, adherence, and performance. Integration with the clinic's electronic medical record (EMR) system may allow session data and progress notes to be documented within existing clinical workflows. A manufacturer or software developer targeting this market may provide an enterprise-grade software license, a multi-seat dashboard for multiple therapists within a practice, and a managed device procurement option, including pre-configured sensor hardware.

In a further implementation, the system may be adapted for use in athletic training rooms, sports medicine facilities, and professional sports organizations as a muscle performance monitoring and injury prevention tool. Rather than targeting post-injury rehabilitation applications, this implementation may be tailored to tracking muscle activation consistency and bilateral balance over time; for example, ensuring that a recovering athlete's injured limb achieves activation parity with the contralateral limb before return to sport is authorized. The system may include multi-channel sensor hardware capable of simultaneous bilateral EMG acquisition, a coaching/training staff dashboard, and individualized athlete performance profiles with trend reporting. Integration with existing sports performance software platforms may allow EMG data to be correlated with other performance metrics.

In yet another implementation, the gamified EMG-based rehabilitation framework may be extended to upper-limb therapeutic applications, including rehabilitation of the forearm, wrist, elbow, and shoulder following injury, surgery, or neurological event. As noted above, the inventors contemplate that the principles and advantages of their MG signal processing and game control pipeline are applicable to other muscle groups beyond the lower limb. Thus, a modular sensor housing (such as in FIG. 6) may be designed for use in adjustable and repositionable sleeves of different sizes and form factors suited to different anatomical areas and body locations. A wearable sleeve or strap designed for the forearm or upper arm may be fitted with surface EMG electrodes targeting relevant muscle groups. The game interface and control mapping may be reconfigured so that upper-limb muscle contractions control distinct game actions, allowing the game to reinforce targeted therapeutic exercises for the upper extremity. This embodiment may be developed and provided by occupational therapy equipment manufacturers, stroke rehabilitation technology developers, and upper-limb orthotics companies.

In a further implementation, the system may be adapted for use in pediatric neurological rehabilitation, addressing conditions such as cerebral palsy, pediatric stroke recovery, or traumatic brain injury affecting motor function. EMG-based sensing may provide a measurement of muscle activation on a standardized motion, allowing providers to track whether muscle recruitment patterns are improving over time, including in cases where overt movement is limited or abnormal. The gamified interface may provide motivating, immediate feedback well-suited to young patients who may have difficulty remaining engaged with traditional exercise-based therapy. The system may be utilized by pediatric hospitals, children's rehabilitation centers, and neurology or developmental medicine practices, and may include integration with hospital EMR systems to surface session data within the care team's existing workflows.

In another implementation, the system may be developed and commercialized as a prescription digital therapeutic (PDT): a software-based therapeutic intervention that may be subject to regulatory review and eligible for third-party reimbursement as a covered digital health benefit. In this embodiment, the system may be marketed to health insurers, employer health benefit programs, and integrated health systems as a clinically-validated, software-delivered rehabilitation intervention. A digital therapeutics company utilizing the systems and methods disclosed herein may obtain approval for a regulated software product incorporating a gamified EMG biofeedback platform, clinical outcome tracking and software or AI-based data interpretation, provider oversight tools, and patient-reported outcome modules.

In a further implementation, the system may be adapted for use in K-12 school settings, such as in health, science, and/or physical education classes (including adaptive physical education (APE) programs) and school-based occupational and physical therapy services. The system may be packaged as a school-friendly kit, including a durable, washable sensor sleeve dimensioned for child sizing, child-safe electrodes, a set of school-appropriate and a simplified educator-facing application interface. Such a kit may allow school-based therapists and adapted PE teachers to deploy the system within a classroom or gym environment, whether for instructional or exercise purposes. The system's educator/therapist dashboard may allow individualized goals to be set for each student, progress to be tracked across multiple sessions, and reports to be generated suitable for inclusion in Individualized Education Program (IEP) documentation.

In yet another implementation, the system may be used for individuals with progressive neuromuscular diseases such as Duchenne muscular dystrophy (DMD) in a maintenance-oriented therapeutic model. Rather than targeting improvement in muscle output, the system may be configured to help patients maintain their current level of function and delay decline. For example, such a system may allow for setting and tracking activation thresholds at a fixed percentage of the patient's baseline maximum, and by adjusting game pacing and obstacle timing to accommodate the easier fatigability in patients with DMD or similar conditions. The system's EMG data capture may also be used to longitudinally track muscle activation patterns, providing objective, quantitative data on functional status that can inform clinical decision-making by a neuromuscular specialist or support endpoints in clinical trials of disease-modifying therapies.

As used in this specification and the claims, the singular forms “a,” “an,” and “the” include plural forms unless the context clearly dictates otherwise.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as,” is intended merely to better illuminate the disclosure and does not pose a limitation on the scope of the disclosure unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, 5, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

1. A method for providing guided therapeutic rehabilitation with improved patient interactivity and compliance, comprising:
accessing stored patient exercise regimen data from a data storage, the patient exercise regimen data comprising at least a first gamification parameter, a first exercise motion, and at least a first EMG sensor position relative to muscles engaged by the first exercise motion;
confirming placement of a wearable EMG device on a patient's limb at the first EMG sensor position;
receiving a real time readout of measurement data from the EMG device;
communicating a digital game environment to a display screen for viewing by a patient, including at least one instruction, the digital game environment having exercise gamification elements configured to influence the patient to perform exercises according to the patient exercise regimen data;
computing change features in the readout of measurement data, and transforming the change features into game control signals corresponding to a patient movement characteristic;
changing at least one game element in the digital game environment according to the game control signal; and
displaying to the patient in real time, using the exercise gamification elements, feedback information comparing performance of the exercises relative to the patient exercise regimen data.

2. The method of claim 1, wherein computing change features in the readout of measurement data comprises:
applying root mean square (RMS) filtering to the readout of measurement data to generate RMS-filtered signals representing muscle activation amplitude; and
applying a moving average filter to the RMS-filtered signals to generate smoothed activation signals, wherein the change features are derived from the smoothed activation signals.

3. The method of claim 1, further comprising, prior to communicating the digital game environment, performing a guided calibration sequence to establish personalized muscle activity parameters for the patient, wherein transforming the change features into game control signals is based at least in part on the personalized muscle activity parameters.

4. The method of claim 3, wherein performing the guided calibration sequence comprises:
instructing the patient to perform a maximum voluntary contraction while wearing the wearable EMG device;
measuring, from the readout of measurement data, a maximum activation value during the maximum voluntary contraction; and
deriving at least one personalized threshold value as a predetermined percentage of the maximum activation value, wherein the at least one personalized threshold value forms at least a portion of the personalized muscle activity parameters.

5. The method of claim 4, wherein changing at least one game element in the digital game environment comprises comparing the game control signals to the at least one personalized threshold value, and selecting between a first game state in which a game character moves in response to the patient's muscle activation and a second game state in which the game character ceases movement.

6. The method of claim 1, further comprising:
storing, in the data storage, session data comprising timestamped values of the readout of measurement data, game control signal values, performance metrics comparing the patient's performance to the patient exercise regimen data, session duration, repetition count, or a combination thereof.

7. The method of claim 6, further comprising:
transmitting the session data to a remote clinical database accessible by an authorized provider for remote monitoring of patient adherence to the patient exercise regimen data.

8. A gamified physical therapy system, comprising,
an EMG device comprising:
at least one EMG sensor configured to sense EMG signals representative of muscle engagement by a user; and
a data link to a processor, to communicate the EMG signals to the processor in real-time;
a wearable article dimensioned to be worn by the user at a first body position, and having a compartment for the EMG device so as to position the EMG device relative to a given target muscle of interest when the wearable article is worn at the first body position; and
a memory comprising a datastore configured to store user data including: a user profile, an exercise regimen, and the EMG signals;
wherein the memory further comprises software instructions which, when executed by the processor, cause the system to:
output an interactive digital game to a user interface including a display, the game having an avatar and game elements defined according to parameters of the exercise regimen;
receive the sensed EMG signals during the game, and compute muscle engagement signals from the sensed EMG signals in real time;
translate the muscle engagement signals into avatar control signals;
depict movement of the avatar in the game on the display in real time according to the avatar control signals, wherein depiction of movement of the avatar is relative to the game elements so as to communicate performance feedback to the user in real time relative to the exercise regimen; and
store in the data store: metadata concerning use of the game; the muscle engagement signals; and data indicative of performance.

9. The gamified physical therapy system of claim 8, wherein the wearable article comprises a plurality of compartments, each compartment configured to house a respective EMG sensor of a plurality of EMG sensors, and wherein the wearable article further comprises one or more labels providing visual indicators to assist in positioning each EMG sensor relative to a corresponding target muscle group when the wearable article is worn at the first body position.

10. The gamified physical therapy system of claim 9, wherein the plurality of EMG sensors are arranged within the wearable article to simultaneously acquire EMG signals from at least two distinct muscle groups, and wherein the software instructions further cause the processor to compute separate muscle engagement signals for each of the at least two distinct muscle groups and to generate comparative activation data between the at least two distinct muscle groups.

11. The gamified physical therapy system of claim 8, wherein the wearable article is formed of an elastic stretch fabric dimensioned to correspond to an average limb circumference at the first body position, and further comprises a hook-and-loop fastener for adjustable closure about such a limb.

12. The gamified physical therapy system of claim 8, wherein the data link comprises a wireless transceiver module configured to wirelessly communicate the EMG signals to the processor, and a microcontroller operatively coupled to the at least one EMG sensor and the wireless transceiver module, the microcontroller configured to digitize and relay the EMG signals to the processor via the wireless transceiver module.

13. The gamified physical therapy system of claim 8, wherein the EMG device further comprises signal conditioning circuitry operatively coupled to the at least one EMG sensor, the signal conditioning circuitry configured to filter time windows of the EMG signals prior to communication to the processor via the data link.

14. The gamified physical therapy system of claim 8, wherein the at least one EMG sensor comprises a plurality of surface electrodes arranged in a bipolar configuration, including a first signal detection electrode, a second signal detection electrode positioned along a longitudinal axis of the wearable article so as to align with a muscle fiber direction of the target muscle of interest, and a reference electrode.

15. The gamified physical therapy system of claim 8, wherein the software instructions further cause the processor to transmit the stored metadata, muscle engagement signals, and data indicative of performance to a remote clinical database accessible by at least one of an authorized clinician, a physical therapist, or an athletic trainer, for remote monitoring of user adherence to the exercise regimen.

16. The gamified physical therapy system of claim 8, wherein the datastore is further configured to store calibration data associated with the user profile, the calibration data comprising a maximum voluntary contraction value measured from the EMG signals during a calibration protocol and at least one personalized threshold value derived as a predetermined percentage of the maximum voluntary contraction value.

17. The gamified physical therapy system of claim 8, wherein the processor is associated with a user device comprising a camera, and wherein the software instructions further cause the processor to:
establish a telehealth session via the camera and a network connection, enabling a remote authorized provider to observe the user performing exercises while wearing the wearable article; and
receive, during the telehealth session, modified parameters of the exercise regimen from the remote authorized provider, and update the game elements of the interactive digital game in accordance with the modified parameters without requiring the user to terminate the game.

18. The gamified physical therapy system of claim 8, wherein the wearable article further comprises at least one inertial measurement unit sensor configured to capture limb orientation and movement data during the game, and wherein the software instructions further cause the processor to correlate the EMG signals with the limb orientation and movement data to distinguish between concentric, eccentric, and isometric contractions performed by the user.

19. The gamified physical therapy system of claim 8, wherein the software instructions further cause the processor to apply a proportional-integral-derivative control algorithm to the muscle engagement signals, wherein proportional, integral, and derivative gain values of the proportional-integral-derivative control algorithm are personalized to the user based on calibration data stored in the datastore, and wherein the avatar control signals are derived at least in part from an output of the proportional-integral-derivative control algorithm.

20. The gamified physical therapy system of claim 8, wherein the software instructions further cause the processor to:
detect, from the muscle engagement signals during the game, a decline in muscle activation amplitude indicative of muscle fatigue; and
in response to detecting the decline, automatically adjust at least one of the game elements or the parameters of the exercise regimen to reduce difficulty or increase rest intervals for a remainder of the game.
